(19) United States
(12) Patent Application Publication
(10) Pub. No.: US 2009/0221893 A1
(43) Pub. Date: Sep. 3, 2009

(54) Unitized Painfree Blood Glucose Measuring Device

(75) Inventor: Terry O. Herndon, Carlisle, MA (US)

Correspondence Address: BANNER & WITCOFF, LTD., 28 STATE STREET, 28th FLOOR, BOSTON, MA, 02109-9601, US

(73) Assignee: PATH SCIENTIFIC, LLC, Carlisle, MA (US)

(21) Appl. No.: 12/394,139

(22) Filed: Feb. 27, 2009

Related U.S. Application Data
(60) Provisional application No. 61/032,486, filed on Feb. 29, 2008.

(51) Int. Cl. A61B 5/00; A61B 5/151

(52) U.S. Cl. 600/365; 600/583

(57) ABSTRACT

A blood glucose measuring device is equipped with a drill device, attachment assembly, and disposable sensing and measurement assembly. The attachment assembly contains an attachment ring that connects to the drill device and is used to hold the disposable sensing and measurement assembly. A detach actuating cam and output shaft are attached to the drill device. Spring tongs are attached to the output shaft by a compression ring further clamp to an end cap. A skin penetrator is attached to the end cap. The disposable sensing and measurement assembly is enclosed in a disposable case. An outer telescoping anti-bend tube is attached to the end cap. An inner anti-bend capillary sensor tube contains analyte sensors and is attached to the disposable case. The electrical conductors for the analyte sensor electrodes are attached to the capillary sensor tube and thus to the disposable case. An impedance sensing electrode on the bottom of the case provides electrical contacts to the skin. The electrical conductor to the impedance sensing electrode is attached to the bottom of the disposable case.

### CROSS-REFERENCE TO RELATED APPLICATION

This application claims priority from U.S. Provisional Application Ser. No. 61/032,486 filed Feb. 29, 2008, the disclosure of which is hereby incorporated by reference.

### BACKGROUND OF THE INVENTION

A number of blood glucose measuring instruments are available with replaceable test strips that are designed to plug-in for one-time use in blood sampling.

U.S. Patent Application Publication No. 20020177763A1 utilizes a method in which the lancet contains a set of electrodes with reagents and a pointed tip. When the lancet pierces the skin, the blood contacts the part of the lancet with the reagent. This particular method does not make use of electrical impedance or control the depth of the penetration.

U.S. Patent Application Publication No. 20050177201A1 uses a method of inserting by driving in with a motor a probe through the skin in small increments so as to minimize pain. The application describes measuring blood glucose and delivering insulin by slowly inserting the probe. This method mostly concerns itself with the pattern of driving the probe into the skin by applying a waveform to the drive motor and does not involve electrical impedance.

U.S. Patent Application Publication No. 20070293747A1 discloses a device that includes a lancet, a capillary tube for drawing out the fluid and a test strip fixed to the end of the capillary tube. This method uses a conventional lancet and does not include any depth control nor use electrical impedance feedback.

U.S. Patent Application Publication No. 20070249963A1 utilizes an integrated device that includes a skin-piercing lancet that acts to draw blood into a compartment where it is analyzed with a reagent. The lancet used is a regular spring activated lancet and hence the pain level will be similar to the already existing lancets.

U.S. Patent Application Publication No. 20060178573A1 describes something similar to mesoscissioning. The sensor used is included in the needle probe. This method employs an integrated electrochemical test strip and does not include an external drive motor.

The aforementioned instruments generally require that a spring-driven lancet driver/retractor is first placed on the skin, followed by the pressing of a trigger which fires the lancet into the skin from which it is then automatically and quickly withdrawn. This produces a sharp pain sensation which is reported to range from slight to intolerable in young people. The lancet pierces the skin to make a sufficiently large and deep puncture wound from which an adequately sized bolus of blood can emerge to fill the tiny capillary on the glucose test strip.

These spring-driven processes generally involve loading a one-time-use lancet into a driver/retractor, then inserting the test strip into the read-out instrument and in some cases calibrating it. The lancet driver is then placed against the skin, fired, and set aside. It is often times necessary to squeeze the lanced site to push out as big a bolus of blood as is needed. The edge of the test strip end, or the side containing the tiny capillary chamber, is held against the blood bolus, where some of it is sucked into the chamber by capillary action. The device notifies the user that the capillary has filled adequately to measure the blood glucose and the test strip can be lifted away from the blood bolus. Incorporated into the device is readout for the display of the automatically calculated glucose level number.

### SUMMARY OF THE INVENTION

The present invention is directed to a blood glucose (or other analyte) measuring device that includes a drill device, an attachment assembly, and a disposable sensing and measurement assembly. The attachment assembly contains an attachment ring that connects to the drill device and is used to hold the disposable sensing and measurement assembly. A detach actuating cam and output shaft are attached to the drill device. Spring tongs are attached to the output shaft by a compression ring further clamp to an end cap. A skin penetrator is attached to the end cap. The disposable sensing and measurement assembly is enclosed in a disposable case. An outer telescoping anti-bend tube is attached to the end cap. An inner anti-bend capillary sensor tube contains analyte sensors and is attached to the disposable case. The electrical conductors for the analyte sensor electrodes are attached to the capillary sensor tube and thus to the disposable case. An impedance sensing electrode on the bottom of the case provides electrical contacts to the skin. The electrical conductor to the impedance sensing electrode is attached to the bottom of the disposable case.

The unitized, painless i.e., pain free or nearly pain free, glucose and other analyte measuring system that is described herein uses the drill device that is described in U.S. patent application Ser. No. 11/206,232, published as U.S. Patent Publication No. 20060041241A1. The disclosure of this application is hereby incorporated by reference.

The drill device of the present invention is modified from its original design for drilling microconduits in nails and removing stratum corneum. The nosepiece 40 is replaced by an attachment assembly and a disposable sensing and measurement assembly. The drill collar 50, receiving bit 70 and feet 55 are removed. The D. C. drill motor is used in only certain embodiments. Also, the foot switch 175 is replaced with a hand-operated switch. In certain embodiments, the D. C. vertical drive motor 80 is replaced by a setting motor for which a stepping power supply and settable counter would replace the vertical drive 185 power supply.

In addition to the drill device modifications described above, the present invention provides an attachment assembly which is attached to the drill device to permit a disposable sensing and measurement assembly to be removably attached to the drill device. In addition, the electronic control circuits of the drill device have been modified to:
(1) start counting the steps once a preset trigger impedance is sensed between the skin penetrator 10 and the electrically conductive impedance sensing electrode 14 on the bottom of the case containing the disposable sensing and measurement assembly,
(2) include a vertical drive motor step counter that can be preset to stop the forward motion of the skin penetrator after reaching the desired count (desired penetrator depth),
(3) include a forward motion rate or speed adjustment capability,
(4) include a reverse rate or speed adjustment capability, and
(5) include an end of reverse motion counter that will stop the reverse motion and reset the system for the next forward cycle.

Additional modifications to the drill device include the electronics, read-out, and inputs from the analyte sensing element of the disposable sensing and measurement assembly.

One preferred embodiment of the present invention is a unitized pain-free blood glucose measuring device comprising a drill device; and an attachment assembly; and a disposable sensing and measurement assembly; wherein the drill device comprises a vertical drive motor to raise and lower an output shaft and further comprises (a) electronics to control the vertical driver motor; and (b) contacts for analyte sensing and impedance sensing electrodes; and (c) a read-out to display analyte concentration; and (d) electrical conductors in contact with impedance sensing electrodes in a disposable sensing and measurement assembly; and (e) electrical conductors in contact with analyte sensing electrodes in the disposable sensing and measurement assembly;

wherein the attachment assembly has a top and bottom, and the output shaft has an upper and lower end and the top of the attachment assembly contacts the drill device at the upper end of the output shaft;

wherein the attachment assembly further includes an assembly attachment ring; and wherein the disposable sensing and measurement assembly is enclosed in a disposable case;

wherein the disposable sensing and measurement assembly has a top and bottom wherein the top is removably attached to the drill device by means of the attachment assembly;

wherein the disposable sensing and measurement assembly further comprises:

an impedance sensing electrode in communication with the electrical contacts in the drill device by means of the assembly attachment ring;

analyte sensor and analyte sensing electrodes in communication with the electrical contacts in the drill device by means of the assembly attachment ring;

an end cap with a top and bottom, wherein the top is attached to the lower end of the output shaft;

a skin penetrator with a puncture end and driver end, wherein the driver end is attached to the bottom of the end cap;

an outer telescoping, anti-bend tube with a top and bottom, wherein the top end is connected to the bottom of the end cap and the tube is centrally positioned around the skin penetrator;

an inner anti-bend capillary sensor tube with a top and bottom,

wherein the top of the inner sensor tube telescopes into the bottom of the outer telescoping, anti-bend tube and the tube is centrally located around the skin penetrator; and

wherein the bottom of the inner anti-bend capillary sensor tube is attached to the bottom of the disposable case.

Preferably, the analyte sensor and sensing electrodes are disposed within the inner anti-bend capillary sensor tube. Preferably, the impedance sensing electrode is an electrically conducting ring peripherally located around a centrally located recess on the bottom of the disposable case. Preferably, the end cap has grooved sides.

In certain preferred embodiments, the unitized blood glucose measuring device further comprises a pair of spring-loaded, outside telescoping, anti-bend tube withdrawal tongs with a first and second end, wherein the second end can clamp to the grooved sides of the end cap.

Preferably, the blood glucose measuring device further comprises a skin penetrator detaching cam with a top and bottom side, wherein the top side is attached to the drill device and the bottom side is removably attached to the first end of the withdrawal tongs.

In certain embodiments, the vertical drive motor is in communication with the output shaft and further comprises electronic control circuits in communication with the drive motor and electrical contacts on the disposable sensing and measurement assembly. Preferably, the vertical drive motor is a stepping motor. Preferably, the electronic control circuits further comprise:

a control mechanism that starts the stepper motor;

a control mechanism that starts counting steps once the electrical impedance between the skin penetrator and the impedance sensing electrode reaches a pre-set trigger impedance value;

a control mechanism that stops the stepper motor once a pre-set step count is reached;

a control mechanism to control a forward motion rate adjustment capability;

a control mechanism to control a reverse rate adjustment capability; and

a control mechanism to control an end of reverse motion trigger that will stop the cycle and pre-set the system for the next forward cycle.

In certain embodiments, the skin penetrator is a stiff, hard wire or fluted shaft of electrically conducting material such as, but not limited to, tungsten or stainless steel. In certain embodiments, the skin penetrator is an acupuncture needle. Preferably, the skin penetrator is coated with a blood wetting material to help draw out fluid behind it as it is withdrawn.

In certain embodiments, a self-aligned petal arrangement of from 3 to 6 protruding petals is attached to the disposable case as an alignment aid. Such petals may be made from stainless steel or an appropriate plastic.

In certain embodiments, the output shaft is rotated by a motorized drive and the rotation is controlled by electronics in the drill device.

In certain embodiments, the inner anti-bend capillary sensor tube is made up of two or more concentric layers.

In certain embodiments, wherein one or more of the layers contain electrodes; the electrodes are in communication with electrical contacts in the drill device by means of the assembly attachment ring.

Another preferred embodiment of the present invention is a method of providing unitized, painless measurement of blood glucose using the unitized blood glucose measuring device described herein. This method comprises the steps of:

attaching the sensing and measurement assembly to the drill device;

setting a preset trigger impedance and a preset step count;

pressing the sensing and measurement assembly against the skin of a user;

activating the skin penetrator to move toward the skin;

starting the step count once the impedance between the skin penetrator and the impedance sensing electrode reaches the preset trigger impedance value;

moving the skin penetrator into the skin to a predetermined step count and stopping;

withdrawing the skin penetrator and allowing fluid to accumulate into the inner capillary sensor tube and contact the analyte sensor and electrodes located therein;

sensing analyte with the analyte sensor and transmitting an electrical signal to the electrical contacts; and

displaying an analyte concentration value to the user.

In certain embodiments, the method further comprises the steps of:

withdrawing the skin penetrator until the output shaft reaches an end position and stops;

releasing the spring-loaded, outer telescoping, anti-bend tube withdrawal tongs holding the end cap; and

removing the disposable sensing and measurement assembly and pulling it away from the unitized blood glucose measuring device.

Preferably, the method further comprises the step of attaching a new sensing and measurement assembly to the drill device.

In certain embodiments, the skin penetrator is inserted into the skin at a rate of speed of about 0.0125 inches per 0.1 seconds.

In certain embodiments, the skin penetrator is retracted from the skin at a rate of speed of about 0.0125 inches in from 10 to 30 seconds.

### BRIEF DESCRIPTION OF THE DRAWINGS

FIG. 1 shows the prior art drilling device, which has been modified for use in the present invention. The reference numbers shown in this drawing are those used in U.S. Patent Publication No. 20060041241A1.

FIGS. 1A and 1B illustrate the attachment assembly of the present invention which contains an assembly attachment ring with a top portion that is fixed to the bottom of the drill device of FIG. 1 and is used to hold the disposable sensing and measurement assembly. FIG. 1A shows the attachment assembly prior to use, and FIG. 1B shows the attachment assembly in the use position, with the skin penetrator engaged with tissue.

FIG. 1C shows the connection of the electrical conductors from the impedance sensing electrode 13 and the analyte sensor electrodes 12 to the drill device through the assembly attachment ring.

FIG. 1D shows the details of the inner sensor tube 9, the bottom of the disposable case 11 with a recess, and conductors from the impedance sensing electrode 14 and the analyte sensing electrodes 12.

### DETAILED DESCRIPTION OF THE PREFERRED EMBODIMENT

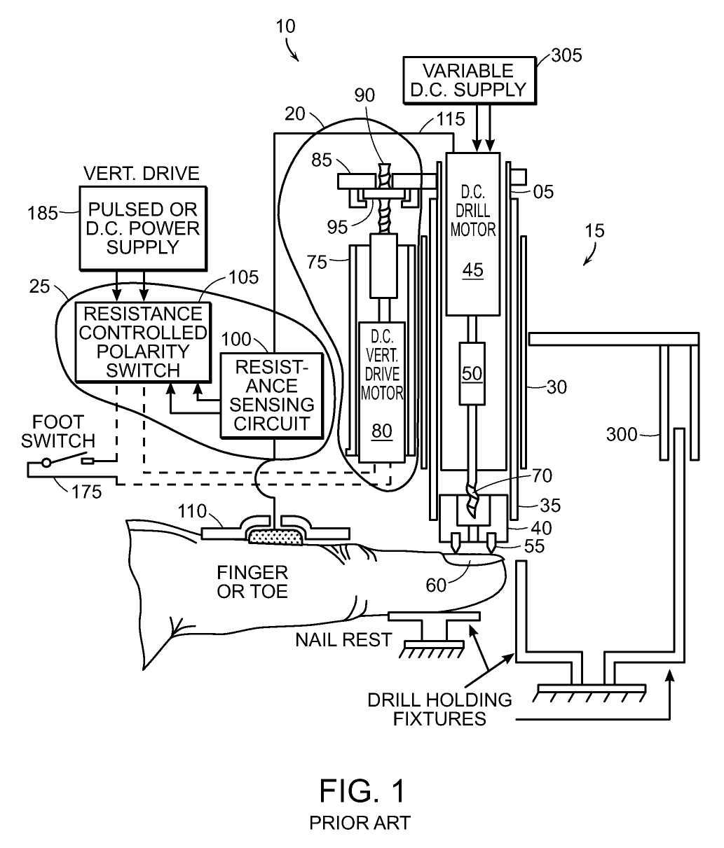

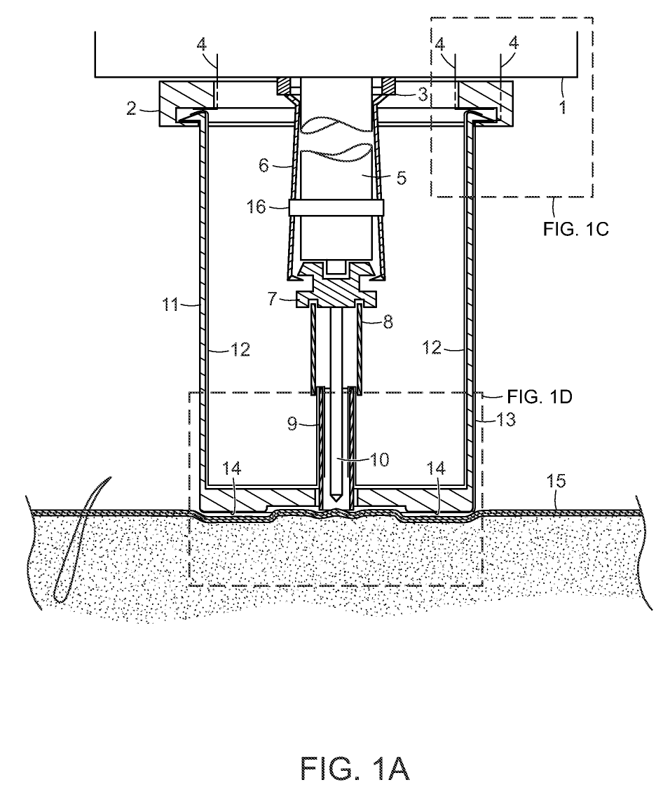

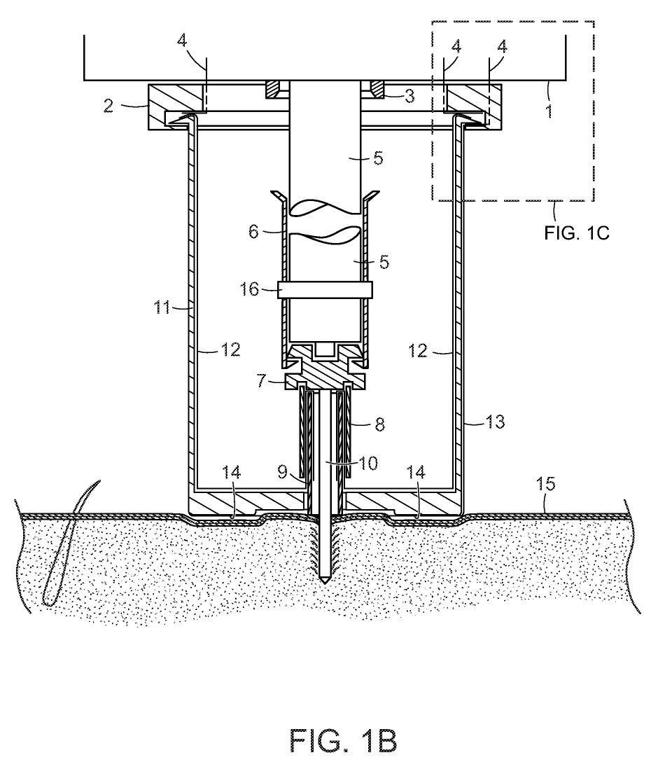

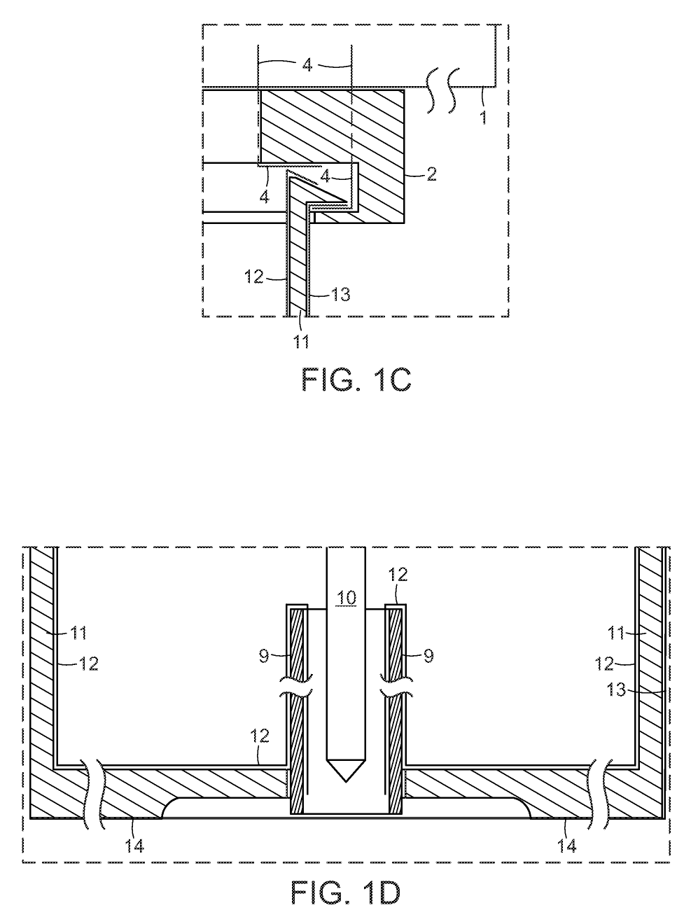

Referring to FIGS. 1A, 1B, 1C and 1D, the attachment assembly contains an assembly attachment ring 2 with a top portion that is fixed to the bottom of the drill device 1 and is used to hold the disposable sensing and measurement assembly. The skin penetrator detach actuating cam 3 is fixed to the drill device 1. The extendible drill device output shaft that has the ability to rotate is 5. Spring tongs 6 attached to the output shaft 5 clamp the end cap 7 to the output shaft 5.

The disposable sensing and measurement assembly is enclosed in a disposable case 11. A disposable end cap 7 is temporarily attached to the drill device output shaft 5. An outer telescoping anti-bend tube 8 is fixed to 7. An inner anti-bend capillary sensor tube 9 that includes an analyte sensor in its lower end is fixed to 11. The impedance sensing electrode 14 on the bottom of the disposable case 11 provides electrical contacts to the skin stratum corneum, and thus to the epidermis and dermis 15. The electrical conductor 13 to the impedance sensing electrode is fixed to 11 on the bottom of the disposable case (FIG. 1D). The spring-loaded, outer telescoping, anti-bend tube withdrawal tongs 6 are fixed to 5 by a compression ring 16. The skin penetrator 10 is fixed to the disposable end cap 7. The electrical conductors for the analyte sensor electrodes 12 are fixed to 9 and thence to 11.

The attachment assembly and the disposable sensing and measurement assembly that is attached to the drill device of FIG. 1 operate as follows:

In FIG. 1A, the disposable sensing and measurement assembly items 7 through 14, contained within the disposable case 11, is snapped into the assembly alignment ring 2 that is attached to the bottom of the drill device 1. The disposable case 11 and assembly attachment ring 2 are rotationally aligned by means of a readily visible, obvious tab-in-slot arrangement (not shown).

Electrical connections from the analyte sensors (located in the inner capillary sensor tube 9) provided by analyte sensing electrodes 12 and electrical connections from the impedance sensing electrode 14 (provided by impedance sensing electrode conductors 13) are connected to the electronic circuits in the drill device 1 with electrical conductors 4 through the assembly attachment ring 2 (FIGS. 1C and 1D). The electrical conductors in the drill device 4 carry the electrical signals from the analyte sensing electrodes 12 and the impedance sensing electrode conductors 13 to appropriate analyte read-out electronics and resistance sensing circuits in the drill device (FIG. 1C). These connections are made through contact pads on matching contacting conductors on the assembly attachment ring 2 (see FIG. 1C).

Attaching the disposable case 11 on the drill device 1 by way of the assembly attachment ring 2 also aligns the drill device 1 output shaft 5 with the end cap 7 in the outer telescoping anti-bend tube 8. The end of the output shaft 5 has a square drive projection. This fits into the square opening in the top of the end cap 7 to permit transmittal of rotational force (if needed) to the end cap. In addition, the end cap 7 has grooved sides, to allow the spring-loaded, outer telescoping, anti-bend tube withdrawal tongs 6 attached to the output shaft 5 by the compression ring 16 to capture the end cap 7. This allows the output shaft to raise the end cap 7, outer telescoping anti-bend tube 8, and skin penetrator 10 during retraction. When the output shaft is retracted, the skin penetrator detach actuating cam 3 attached to the drill device 1 contacts the withdrawal tongs and spreads them out from the end cap 7, releasing it to permit the removal of the disposable case 11 containing the sensing and measurement assembly.

In combination, the outer telescoping anti-bend tube 8 and the inner anti-bend capillary sensor tube 9 serve to keep the skin penetrator 10 from buckling or bending as the output shaft 5 moves down, driving the end cap 7 down to push the skin penetrator 10 into the skin. The output shaft 5 and hence the skin penetrator 10 may be stationary or rotating when inserted or withdrawn.

As shown in FIG. 1B, with the inner anti-bend capillary sensor tube 9 snugly against the skin 15, the skin penetrator 10 acts as a piston in a cylinder of skin and creates a vacuum. This acts in conjunction with the mechanical pressure created in the tissues around the site by the recess in the case 11 to assist in extracting blood directly from the tissue into the inner anti-bend capillary sensor tube 9 which also contains the analyte sensor. The impedance sensing electrode is preferably a ring in shape but is not restricted to this shape. The height of the recess (FIG. 1D) in the bottom of the case 11 with the impedance sensing electrode 14 is designed to maximize the blood flow resulting from the higher downward pressure produced in the skin by forming a thicker, outer ring that presses more deeply into the tissue around the site pierced by the skin penetrator 10.

Inserting the skin penetrator 10 at a high rate of speed, in the range of 0.0125 inches per 0.1 second, greatly minimizes the sensation level. Removal of the skin penetrator 10 at a rate of 0.0125 inches in 10-30 seconds maximizes the volume of blood extracted. Faster removal rates tend to produce little or no blood yield, while slower rates produce no additional blood.

Before operating the device, the protective adhesive tape cover applied across the top of the disposable case 11 must be removed by pulling on its marked tab (not shown). The disposable case tabs are aligned with slots in the assembly attachment ring 2, rotated and attached to the drill device 1. The protective adhesive tape cover across the bottom of the disposable case 11 is removed by pulling on its tab (not shown). The drill device is placed firmly against the skin and is activated. The rate of skin penetration by the penetrator 10 may be varied to minimize time and/or sensation. The depth of penetration of the skin penetrator 10 can be pre-set with a step count in the drill device 1 down-drive stepper motor control electronics. The start count signal arises when the penetrator 10 pierces the high electrical resistance of the stratum corneum on the outer surface of the skin 15 and the electrical impedance between 10 and 14 drops. The skin penetrator 10 moves into the skin until it reaches the predetermined depth and stops. The withdrawal of the skin penetrator 10 from the tissue begins automatically and the rate may be varied to maximize the outward blood flow.

As the skin penetrator 10 is withdrawn back into the inner anti-bend capillary sensor tube 9, blood follows out with it, drawn in by the blood-philic nature of the penetrator 10, by capillary action, and by pressure in the tissue from the device pressing against a ring of the skin. This is aided by the vacuum produced by the withdrawing skin penetrator 10 in the inner anti-bend capillary sensor tube 9.

The inner anti-bend capillary sensor tube 9 contains the analyte sensor and the sensing electrodes printed on its inner surface. When sufficient blood covers the sensor, the chemicals in the sensor cause a change in charge, current, or optical signal which is further communicated by the analyte sensing electrodes 12 to the drill device 1. The device electronics translate the electrical signal into a corresponding analyte concentration value displayed on a visible readout on the outer surface of the drill device 1.

The skin penetrator 10 continues to withdraw until the drill device 1 output shaft 5 reaches the end position and stops. This then raises the top ends of the spring tongs 6 to engage the skin penetrator detach actuating cam 3 that opens the spring-loaded, outer telescoping, anti-bend tube withdrawal tongs 6 to release the end cap 7. The disposable case 11 can then be rotated and pulled away from the drill device 1 and discarded.

In one embodiment, the skin penetrator 10 is a stiff, hard wire made of electrically conducting materials such as tungsten or stainless steel. Depending on the desired use, the tip can be ground into any configuration from flat to pointed. The disposable end cap 7 is cast around the skin penetrator 10 to ensure an inseparable joint. The skin penetrator 10 surface may be microroughened or smoothed or contain straight or curved microgrooves and may be coated with such materials as heparin to reduce clotting of the blood to maximize the blood flow. In addition, the penetrator 10 may be coated with a blood wetting material to help draw the blood out behind it as it is withdrawn. Blood wetting materials are commercially available to the skilled artisan.

In another embodiment, an acupuncture needle, approximately 0.12 mm (0.0047 inch) in diameter or larger, can be used as a highly polished, very sharp skin penetrator 10. This can be inserted 0.0125 inches or more into the skin with only a very slight pricking sensation with no sensation during withdrawal from the skin.

For use on fingertips, an alignment aid can be provided by a using a self-aligned petal arrangement, containing 3-6 down-protruding petals (not shown) which is first snapped onto the bottom of the disposable case before use.

The present invention has been described in detail, including the preferred embodiments thereof. However, it will be appreciated that those skilled in the art, upon consideration of the present disclosure, may make modifications and/or improvements on this invention and still be within the scope of this invention as set forth in the following claims.

What is claimed is:

1. A unitized pain-free blood glucose measuring device comprising:
a drill device;
an attachment assembly; and
a disposable sensing and measurement assembly;
wherein the drill device comprises a vertical drive motor to raise and lower an output shaft and further comprises:
electronics to control the vertical driver motor;
contacts for analyte sensing and impedance sensing electrodes;
a read-out to display analyte concentration;
electrical conductors in contact with impedance sensing electrodes in a disposable sensing and measurement assembly;
electrical conductors in contact with analyte sensing electrodes in the disposable sensing and measurement assembly; and
wherein the attachment assembly has a top and bottom, and the output shaft has an upper and lower end and the top of the attachment assembly contacts the drill device at the upper end of the output shaft;
wherein the attachment assembly further includes an assembly attachment ring;
wherein the disposable sensing and measurement assembly is enclosed in a disposable case;
wherein the disposable sensing and measurement assembly has a top and bottom wherein the top is removably attached to the drill device by means of the attachment assembly;
wherein the disposable sensing and measurement assembly further comprises:
an impedance sensing electrode in communication with the electrical contacts in the drill device by means of the assembly attachment ring;
analyte sensor and analyte sensing electrodes in communication with the electrical contacts in the drill device by means of the assembly attachment ring;
an end cap with a top and bottom, wherein the top is attached to the lower end of the output shaft;
a skin penetrator with a puncture end and driver end, wherein the driver end is attached to the bottom of the end cap;
an outer telescoping, anti-bend tube with a top and bottom, wherein the top end is connected to the bottom of the end cap and the tube is centrally positioned around the skin penetrator;
an inner anti-bend capillary sensor tube with a top and bottom,
wherein the top of the inner sensor tube telescopes into the bottom of the outer telescoping, anti-bend tube and the tube is centrally located around the skin penetrator; and
wherein the bottom of the inner anti-bend capillary sensor tube is attached to the bottom of the disposable case.

2. The unitized blood glucose measuring device of claim 1, wherein the analyte sensor and sensing electrodes are disposed within the inner anti-bend capillary sensor tube.

3. The unitized blood glucose measuring device of claim 1, wherein the impedance sensing electrode is an electrically conducting member peripherally located around a centrally located recess on the bottom of the disposable case.

4. The unitized blood glucose measuring device of claim 1, wherein the end cap has grooved sides.

5. The unitized blood glucose measuring device of claim 4, further comprising a pair of spring-loaded, outside telescoping, anti-bend tube withdrawal tongs with a first and second end, wherein the second end can clamp to the grooved sides of the end cap.

6. The unitized blood glucose measuring device of claim 5 further comprising a skin penetrator detaching cam with a top and bottom side, wherein the top side is attached to the drill device and the bottom side is removably attached to the first end of the withdrawal tongs.

7. The unitized blood glucose measuring device of claim 1, wherein the vertical drive motor is in communication with the output shaft and further comprises electronic control circuits in communication with the drive motor and electrical contacts on the disposable sensing and measurement assembly.

8. The unitized blood glucose measuring device of claim 7, wherein the vertical drive motor is a stepping motor.

9. The impedance sensing assembly of claim 8, wherein the electronic control circuits further comprise:
a control mechanism that starts the stepper motor;
a control mechanism that starts counting steps once the electrical impedance between the skin penetrator and the impedance sensing electrode reaches a preset trigger impedance value;
a control mechanism that stops the stepper motor once a pre-set step count is reached;
a control mechanism to control a forward motion rate adjustment capability;
a control mechanism to control a reverse rate adjustment capability; and
a control mechanism to control an end of reverse motion trigger that will stop the cycle and pre-set the system for the next forward cycle.

10. The unitized blood glucose measuring device of claim 1, wherein the skin penetrator is a stiff, hard wire or fluted shaft of electrically conducting material such as, but not limited to, tungsten or stainless steel.

11. The unitized blood glucose measuring device of claim 10, wherein the skin penetrator is coated with a blood wetting material to help draw out fluid behind it as it is withdrawn.

12. The unitized blood glucose measuring device of claim 1, wherein the skin penetrator is an acupuncture needle.

13. The unitized blood glucose measuring device of claim 12, wherein the skin penetrator is coated with a blood wetting material to help draw out fluid behind it as it is withdrawn.

14. The unitized blood glucose measuring device of claim 1, wherein a self-aligned petal arrangement of from 3 to 6 protruding petals is attached to the disposable case as an alignment aid.

15. The unitized blood glucose measuring device of claim 1, wherein the output shaft is rotated by a motorized drive and the rotation is controlled by electronics in the drill device.

16. The unitized blood glucose measuring device of claim 1, wherein the inner anti-bend capillary sensor tube is made up of two or more concentric layers.

17. The unitized blood glucose measuring device of claim 16, wherein one or more of the layers contain electrodes; wherein the electrodes are in communication with electrical contacts in the drill device by means of the assembly attachment ring.

18. A method of unitized, painless measurement of blood glucose using the unitized blood glucose measuring device of claim 5, comprising the steps of:
attaching the sensing and measurement assembly to the drill device;
setting a preset trigger impedance and a preset step count;
pressing the sensing and measurement assembly against the skin of a user;
activating the skin penetrator to move toward the skin;
starting the step count once the impedance between the skin penetrator and the impedance sensing electrode reaches the preset trigger impedance value;
moving the skin penetrator into the skin to a predetermined step count and stopping;
withdrawing the skin penetrator and allowing fluid to accumulate into the inner capillary sensor tube and contacting analyte sensor and electrodes located therein;
sensing analyte with the analyte sensor and transmitting an electrical signal to the electrical contacts; and
displaying an analyte concentration value to the user.

19. The method of claim 18, further comprising the steps of:
withdrawing the skin penetrator until the output shaft reaches an end position and stops;
releasing the spring-loaded, outer telescoping, anti-bend tube withdrawal tongs holding the end cap; and
removing the disposable sensing and measurement assembly and pulling it away from the unitized blood glucose measuring device.

20. The method of claim 19, further comprising the step of attaching a new sensing and measurement assembly to the drill device.

21. The method of claim 20, wherein the skin penetrator is inserted at a rate of speed of about 0.0125 inches per 0.1 seconds.

22. The method of claim 20, wherein the skin penetrator is retracted at a rate of speed of about 0.0125 inches in from 10 to 30 seconds.

23. A blood glucose measuring device comprising
(a) a drill device,
(b) an attachment assembly, and
(c) a disposable sensing and measurement assembly;
wherein the attachment assembly contains an attachment ring that connects to the drill device and is used to hold the disposable sensing and measurement assembly;
(d) a detach actuating cam and output shaft are attached to the drill device;
(e) spring tongs are attached to the output shaft by a compression ring further clamp to an end cap;
(f) a skin penetrator is attached to the end cap;
(g) the disposable sensing and measurement assembly is enclosed in a disposable case;
(h) an outer telescoping anti-bend tube is attached to the end cap;
(i) an inner anti-bend capillary sensor tube contains analyte sensors and is attached to the disposable case;
(j) electrical conductors for the analyte sensor electrodes are attached to the capillary sensor tube and thus to the disposable case;
(k) an impedance sensing electrode on the bottom of the case provides electrical contacts to the user's skin; and
(l) an electrical conductor to the impedance sensing electrode is attached to the bottom of the disposable case.
